(19) United States
(12) Patent Application Publication
(10) Pub. No.: US 2010/0116276 A1
(43) Pub. Date: May 13, 2010

(54) VENTILATION MASK

(76) Inventor: Mohamed Ghiath Bayasi, Troy, MI (US)

Correspondence Address: REISING ETHINGTON P.C., P O BOX 4390, TROY, MI, 48099-4390, US

(21) Appl. No.: 12/354,263

(22) Filed: Jan. 15, 2009

Related U.S. Application Data
(60) Provisional application No. 61/112,422, filed on Nov. 7, 2008.

(51) Int. Cl. A61M 16/06

(52) U.S. Cl. 128/207.12; 128/207.13

(57) ABSTRACT

A ventilation mask is provided. The ventilation mask comprises a body having a seal on a peripheral edge. The ventilation mask includes an access aperture having a valve associated therewith. The valve is selectively moveable between an open position, allowing objects to be place through the body and a closed position. In the closed position, the valve minimizes or prevents air leakage through the access aperture. The access aperture allows procedures to be performed on a wearer of the mask, without interrupting air flow to the wearer.

### CROSS-REFERENCE TO RELATED APPLICATION

This application claims the benefit of U.S. Provisional Application No. 61/112,422, filed Nov. 7, 2008, the disclosure of which is incorporated herein.

### FIELD OF THE INVENTION

The present invention relates generally relates to a ventilation mask. More specifically, the present invention relates to a non-invasive ventilation mask having access openings.

### BACKGROUND OF THE INVENTION

Patients who suffer from respiratory failure require positive pressure mechanical ventilation. Positive pressure mechanical ventilation can be provided with either an invasive endobronchial tube or a non-invasive tight fit mask. Accordingly, tight fit masks, such as oronasal masks eliminate the need for intubation or tracheostomy.

Oronasal masks while having certain advantages over endobronchial tubes have certain deficiencies. For example, in some instances nasogastric tubes or Bronchoscopies may also be required by a user (or patient). However, the use of such a tube requires it to be placed adjacent the plastic rim of the oronasal mask that can result in breaking the seal between the mask and the user's face and cause a detrimental air leak. Other deficiencies may arise resulting in conditions such as mouth and pharynx dryness causing infections or occasionally mucous plugs in the user. Other upper airways conditions associated with the use of an oronasal mask may include nasal stuffiness, runny nose, bloody nose, or other difficulty managing oral secretions. These conditions often require treatment from a caregiver to be rectified. Some user's medical conditions do not allow the mask to be removed to deliver necessary oral or nasal care. Further, caring for patients requiring such oronasal masks often requires considerable time. Access to the patient's mouth or nose is restricted due to the undesirability of or inability to remove the mask. The lack of sufficient time for a caregiver to devote to some patients may exacerbate the conditions needed to be treated.

Accordingly, it may be desirable to provide a tight-fit mask, such as an oronasal mask that can be used to provide positive pressure ventilation and provides accessibility to the patient's mouth or nose without adversely affecting the use of the mask for ventilation.

### SUMMARY OF THE INVENTION

According to an embodiment, there is provided a ventilation mask comprising a body defining an access aperture therethrough. The mask further comprises a valve associated with said access aperture and moveable between an open position to allow objects to pass through said access aperture and a closed position to close said access aperture.

According to an embodiment, there is provided method of using a ventilation mask. The method comprises positioning a mask over a user's mouth and nose. The mask has a valve that is moveable between an open position to allow objects to pass through an access aperture and a closed position to close said access aperture. Air is introduced to the mask through an air delivery aperture to achieve positive pressure ventilation. A valve is opened to allow objects to be inserted through the valve and the access aperture to perform care. The valve is then closed to close the access aperture and prevent air leakage through the access aperture.

### BRIEF DESCRIPTION OF THE DRAWINGS

The present invention will become more fully understood from the detailed description and the accompanying drawings, wherein:

FIG. 1 is a front view of an embodiment of a mask;

FIG. 2 is a side view of an embodiment of a mask; and

FIG. 3 is a perspective view of an embodiment of a mask.

### DETAILED DESCRIPTION OF THE PREFERRED EMBODIMENTS

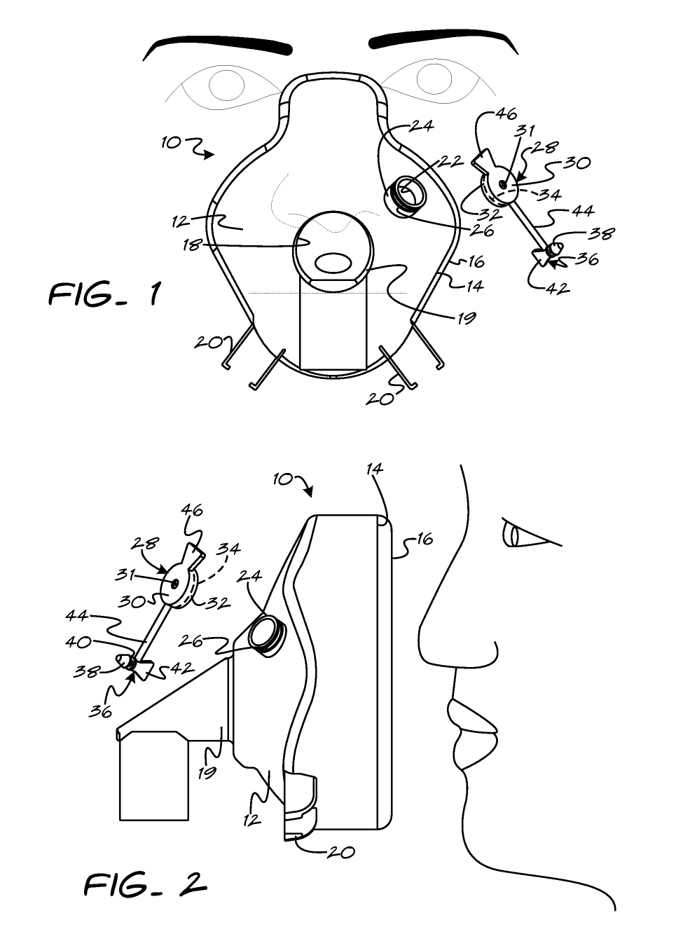

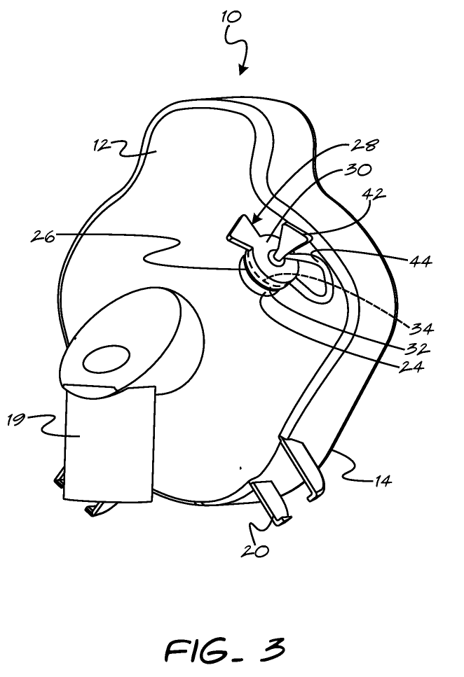

FIG. 1 is a front view of an oronasal mask, generally shown at 10. FIG. 1 shows a valve 28 in an open or accessible position. FIG. 2 is a side view of an oronasal mask 10. FIG. 3 is a perspective view of an oronasal mask 10 showing the valve 28 in a closed or inaccessible position. The mask 10 may take any suitable configuration that can be used to deliver positive pressure ventilation for a user. In an embodiment, the mask 10 covers a user's nose and mouth. The mask 10 is non-invasive and does not require intubation of the user. Non-invasive positive pressure ventilation can be given by any suitable device (not shown), such as by way of non-limiting examples, volume ventilators, pressure controlled ventilators, bi-level positive airway pressure devices or continuous positive airway pressure devices.

The mask 10 is preferably formed of clear plastic material and has a body 12. The body 12 defines a peripheral edge 14 that contacts the user's face. Preferably, the rest of the body 12 extending from the peripheral edge 14 may be spaced from the user's face. The peripheral edge 14 may include a seal 16 thereon to provide a seal between the mask 10 and the user's face. Any suitable seal 16, such as those known in the art, may be used.

The mask 10 may include an air delivery aperture 18 therethrough for connection of an air supply tube (not shown) of a device for delivering positive pressure ventilation. More specifically, air delivery aperture 18 may include a connection segment 19 that can be secured to an air supply tube of a device for delivering positive pressure ventilation (not shown) as is well-known.

The body 12 of the mask 10 may also include one or more connecting flanges 20. The connecting flanges 20 may receive straps (not shown) used to secure the mask 10 on the user's face. The connecting flanges 20 may be placed at any suitable location on the body 12. The use of such straps is well-known. While flanges 20 are shown to receive straps, it will be appreciated that any other suitable manner of securing the mask 10 about the user's nose and mouth is contemplated. By way of non-limiting example the flanges 20 may receive buckles or other devices which, in turn, receive straps. Alternatively buckles or other devices may be integrated into the body 12 for receiving straps or otherwise securing the mask 10.

The body 12 of the mask 10 may further include one or more access apertures 22. The access aperture 22 may take any suitable configuration. As shown, the access aperture 22 is generally circular. The access aperture 22 allows objects to pass through the body 12 without the need to remove the mask 10. This allows a caregiver to provide appropriate care to the user without interrupting the supply of air to the user.

Each access aperture 22 may be bounded by a tubular portion 24. The tubular portion 24 may extend from the body 12 in a direction generally away from the user's face. The tubular portion 24 may take any suitable configuration. As is shown, the tubular portion 24 has a generally circular cross-section. The tubular portion 24 may include an annular fastening groove 26. The access aperture 22 and tubular portion provide an opening allowing access to the interior of the mask. That is, the interior referred to is the area between the body 12 of the mask and the user's face, when the mask 10 is properly positioned, is accessible.

The access aperture 22 and the tubular portion 24 may be sized to allow medical devices, such as by way of non-limiting example, tubes, syringes or bronchoscopes to be inserted to the interior of the mask to perform therapeutic or diagnostic tasks. These tasks may include, but are not limited to oral care of the patient, nasal care of the patient or bronchoscopies. Further the opening may allow tubes, such as nasogastric tubes to be used.

In order to reduce the risk of air leak from the access apertures 22, a valve, generally indicated at 28 and best seen in FIGS. 1 and 3 may be provided. The valve 28 is associated with the access aperture 22 and is moveable between an open position (FIG. 1) and a closed position (FIG. 4). In the open position, the valve 28 allows objects to pass through the body 12. When in the closed position, the valve 28 prevents objects from passing through the body 12 and also closes the access aperture 22 to eliminate air loss therethrough. Thus, the valve 28 may be used to selectively allow access to the interior of the mask 10 in an open position and also to seal the mask 10 in a closed position.

The valve 28 may include a base 30. The base 30 may have an aperture 31 therethrough. An annular wall 32 may extend about the base 30 and is adapted to co-act with the tubular portion 24. More specifically, the annular wall 32 may include a lip 34 that can be retained in the annular fastening groove 26 of the tubular portion 24.

The valve 28 may further include a plug generally indicated at 36 for selectively closing the aperture 31. The plug 36 may include a frustoconical portion 38 having a contact wall 40 at one end of the frustoconical portion. The frustoconical portion 38 and contact wall 40 may be dimensioned so that the plug 36 can be inserted through the aperture 31 to close the aperture 32. Preferably the contact wall 40 includes a generally flat surface that can be used to engage the base 30.

In one embodiment, the valve 28 may be made of any suitable resilient material. The base 30 and annular side wall 32 may be flexible so that the annular side wall 32 can be positioned about the tubular portion 24 and so that the lip 34 can be snap-fit into the annular fastening groove 26 of the tubular portion 24. Similarly, the base 30 allows for insertion of the plug 36, which may also be made of a resilient material, through the aperture 31. Accordingly, the base 30 and or the plug 36 may flex in response to a force applied by the frustoconical portion 38 to open the aperture 31 sufficiently to allow the plug 36 to be inserted through the aperture 31. The frustoconical portion 38 may act as a cam surface to expand the aperture 31. It will be appreciated that the contact wall 40 may be sized so that it has a larger diameter than the diameter of the aperture 31. This allows a seal to be made between the contact wall 40 and the base 30. The positive air pressure during use of the mask 10 will urge the contact wall 40 into engagement with the base 30. Further, it is preferred that the contact wall 40 be sized so that upon application of a suitable force, the plug 36 may be removed from the aperture 31.

The plug 36 may also be secured to a tab 42. The tab 42 allows for a caregiver to grasp it and thereby exert a force on the plug 36. The tab 42 can be used to apply an insertion force—to allow the plug 38 to be inserted through the aperture 31 and thereby close the valve 28. Similarly, the tab 42 may be used to apply an extraction force—to pull the plug 38 through the aperture 31 outwardly to open the aperture 31 and thereby open the valve 28 to provide access to the interior of the mask 10.

The valve 28 may also include a suitable tether 44. The tether 44 may be used to secure the body 30, plug 36 and tab 42. In this manner, the valve 28 may preferably comprise a one-piece unitary member that includes the body 30, plug 36, tab 42 and tether 44. It will be appreciated, however, that the valve 28 may be formed of multiple pieces secured together. Further, the body may also include a second tab 46 on the annular side wall 32. This second tab preferably is integrally formed as one-piece with the valve 28. This second tab 46 may be used to facilitate removal of the valve 28 from the tubular portion 24 by providing an area that a caregiver can grasp.

A valve 28 of the type described herein are known in the industry. Further, while one type of valve 28 is disclosed, it will be appreciated that any suitable valve can be used. It is preferred that the valve 28 allow selective access to the interior of the mask 10. That is, when the valve 28 is in an open position, objects can pass through the body 12. It is also preferred that the valve be moveable to a closed position to prevent objects from passing thought the body 12 and also minimizing air leakage.

The aperture 31 is preferably sized so that it can receive various objects, such as tubes, syringes or bronchoscopes. When the plug 36 is removed from the aperture 31, a tube or other object (not shown) can be placed through the aperture 31. Preferably, the interior edge of the base 30 that defines the aperture 31 may contact the tube or other object to minimize air leakage. The tube or other object can be used to provide temporary oral or nasal care. Upon completion of the task, the tube or other object is removed and the plug 36 can be inserted through the aperture to close the valve 28. Alternatively, the aperture 31 provides an access to allow a more permanent object, such as a nasogastric tube to pass trough the mask 10. The base 30 can contact such a tube and provide a seal to minimize air leakage.

In order to facilitate care giving, the access aperture 22 and tubular portion 24 may preferably be oriented on the body 12 such that they aid it providing care to the patient. That is, the access aperture 22 and tubular portion are preferably angled such that when an object is placed therethrough it may be directed to the user's mouth or nose. By having the access aperture 22 and tubular portion 24 angled in this manner, any tube, or other object entering through the access aperture 22 will most preferably be directed toward the user's mouth or nose.

It will be appreciated that while one access opening 22 is shown in the drawings, any number of access openings 22 may be used. Similarly, a valve 28 may be supplied for each access opening.

In use of the mask 10, the mask 10 is placed on a user such that the seal 16 on the peripheral edge 14 is disposed about the user's mouth and nose. The mask 10 is secured to the user by any suitable means such as suitable stops. Preferably the mask 10 may be tightly fit to the user. The access aperture 22 may be closed by valve 28. This may be achieved by placing the lip 34 into the annular fastening groove 26. The valve 28 may be placed in the closed position by inserting the plug 36 through the aperture 31. Positive pressure ventilation is then achieved by introducing air or oxygen into the interior of the mask 10 through the delivery aperture 18. The positive pressure urges the contact wall 40 with the base 30 to minimize or eliminate air leakage through the valve 28.

When desired, access to the interior of the mask 10 can be achieved to perform tasks, such as oral or nasal care. In order to access the interior of the mask 10, a caregiver may open the valve by grasping the tab 32 and removing the plug 36 from the aperture 31. A tube or other object (not shown) may be inserted through the aperture 31. Preferably, the tube or other object has a diameter close to or greater than that of the diameter of the aperture 31. In this manner, the base 30 may engage the tube or other object and provide a seal to minimize air leakage during any procedure. The tube or other object is aided in being directed to the user's mouth or nose due to the orientation of the access opening 22 and tubular portion 24.

Upon completion of any operation, the caregiver may close the valve 28. To accomplish this, the caregiver extracts the tube or other object from the access aperture 22 trough the aperture 31. The tab 42 is grasped and the plug 36 is inserted through the diameter of the aperture 31 until the contact wall 40 passes through the aperture 31. The contact wall 40 engages the base 30 to provide a seal.

It will be appreciated that in some applications, the tube or other object may need to remain for a relatively longer duration. For example, a nasogastric tube may remain for a relatively longer time to facilitate care for the user.

While the mask 10 described above is in connection with an oronasal mask, it will be appreciated that any type of non-invasive ventilation mask may be used. By way of non-limiting examples, a nasal mask or a full face mask may also be used. In each type of mask, the access openings may include any suitable valve thereon for allowing selective access to the interior of the mask and selective closing of the valve to restrict access to the interior of the mask and to minimize air leakage.

The foregoing description is considered illustrative only. The terminology that is used is intended to be in the nature of words of description rather than of limitation. Many modifications and variations will readily occur to those skilled in the art in view of the description. Thus, the foregoing description is not intended to limit the invention to the embodiments described above. Accordingly the scope of the invention as defined by the appended claims.

What is claimed is:

1. A ventilation mask comprising:
a body defining an access aperture therethrough;
a valve associated with said access aperture and moveable between an open position to allow objects to pass through said access aperture and a closed position to close said access aperture.

2. A ventilation mask as set forth in claim 1 wherein said valve further comprises a base having an aperture therethrough and a plug for selectively closing said aperture.

3. A ventilation mask as set forth in claim 2 wherein said plug is generally frusotconical and includes an annular wall at one end thereof.

4. A ventilation mask as set forth in claim 3 wherein said base is made of a resilient material.

5. A ventilation mask as set forth in claim 2 wherein said valve includes an annular side wall having a lip thereon.

6. A ventilation mask as set forth in claim 5 wherein said body includes a tubular portion defining a groove thereon, said lip disposed in said groove to retain said valve on said tubular portion.

7. A ventilation mask as set forth in claim 6 wherein said tubular portion and said access opening are oriented at an angle such that when an object is placed therethrough it is generally directed at a user's mouth or nose.

8. A ventilation mask as set forth in claim 2 wherein said body defines a peripheral edge, a seal is disposed on said peripheral edge.

9. A ventilation mask as set forth in claim 2 wherein said body defines an oronasal mask.

10. A ventilation mask as set forth in claim 2 wherein said body defines a nasal mask.

11. A ventilation mask as set forth in claim 2 wherein said body defines a full face mask.

12. A method of using a ventilation mask comprising:
positioning a mask over a user's mouth and nose, the mask having a valve that is moveable between an open position to allow objects to pass through an access aperture and a closed position to close said access aperture;
introducing air to the mask through an air delivery aperture to achieve positive pressure ventilation;
opening the valve to allow objects to be inserted through the valve and the access aperture to perform care; and
closing the valve to close the access aperture and prevent air leakage through the access aperture.
